(19) United States
(12) Patent Application Publication
(10) Pub. No.: US 2006/0246454 A1
(43) Pub. Date: Nov. 2, 2006

(54) Influence of genotype on susceptibility to treatment with fish oil

(76) Inventors: Robert Francis Grimble, Southampton (GB); Philip Charles Calder, Southampton (GB); William Martin Howell, Southampton (GB)

Correspondence Address: NIXON & VANDERHYE, PC, 901 NORTH GLEBE ROAD, 11TH FLOOR, ARLINGTON, VA, 22203, US

(21) Appl. No.: 10/553,995

(22) PCT Filed: Apr. 21, 2004
(86) PCT No.: PCT/GB04/01721

§ 371 Date: Jun. 20, 2006

Related U.S. Application Data
(60) Provisional application No. 60/464,336, filed on Apr. 22, 2003.

(51) Int. Cl. C12Q 1/68

(52) U.S. Cl. 435/6

(57) ABSTRACT

The present invention relates to methods for assessing an individual's susceptibility to treatment of an inflammatory disease with a dietary supplement. The invention describes the recognition that the sensitivity of an individual to the inflammation suppressing effects of fish oil on TNF-α production is linked both to genetic variation encoded by, or associated with, TNF-α, LT-α and/or IL-6 single nucleotide polymorphisms and to the inherent level of production of TNF-α by cells.

### FIELD OF THE INVENTION

The present invention relates to methods for assessing an individual's susceptibility to treatment of an inflammatory disease with a dietary supplement.

### BACKGROUND TO THE INVENTION

Inflammation in the human body is mediated by tumour necrosis factor-α (TNF-α). TNF-α is one of a group of pro-inflammatory cytokines which appear rapidly following infection and injury (Beutler et al., Crit Care Med 21:423-35 (1993) and Lin et al., Surgery 127:117-26 (2000)). TNF-α has widespread effects: it causes loss of lean and adipose tissue, raises body temperature, reduces appetite and stimulates production of a diverse range of immunomodulatory cytokines and oxidant molecules (Grimble, Clin Sci 91:121-30 (1996)). These effects create a hostile environment for invading pathogens, provide substrate for the immune system from endogenous sources and enhance and modify the activity of the immune system. Thus TNF-α has a pivotal role in allowing the body to withstand pathogenic invasion.

However, excessive or untimely TNF-α plays a major part in mortality and morbidity from sepsis (van der Poll et al., Infect Dis Clin North Am. 13:413-26 (1999)), meningitis (Westendorp et al., Lancet 349:170-3 (1997)) and malaria (Knight et al., Proc Assoc Am Physicians 111:290-8 (1999)). TNF-α also plays an important part in the pathology of inflammatory diseases such as rheumatoid arthritis (Maini et al., Ann Rev Med 51:207-29 (2000)) and inflammatory bowel disease (Murch et al., Gut 32:913-7 (1991)), in the development of atherosclerotic plaques (Ross, Nature 362:801-9 1993)) and in rejection of transplanted tissues (Kutukculer et al., Transpl Int. 8:45-50 (1995)).

Fish oil, which is rich in n-3 polyunsaturated fatty acids (n-3 PUFAs) has been shown to exert an anti-inflammatory influence in a number of animal models of inflammation (Calder, Ann Nutr Metab 41:203-34 1997) and Grimble, Proc Nutr Soc. 57:535-42 1998)) and produces anti-inflammatory effects in rheumatoid arthritis (Calder & Zurier Curr Opin Clin Nutr Metab Care 4:115-21 (2001)), Crohn's disease (Belluzi et al., N Engl J Med 334:1557-616 (1996)) and psoriasis (Mayser et al., J Am Acad Dermatol 38:539-47 (1998)). Encapsulated fish oils are often taken as a dietary supplement.

One of the reported anti-inflammatory actions of fish oil is in a reduction in the production of TNF-α by peripheral blood mononuclear cells (PBMC) (Endres et al., N Engl J Med 320:265-71 (1989)). However, in studies in which fish oil has been investigated as an anti-inflammatory agent, it has not been found to be effective in all individuals. For example, of eleven studies investigating the effect of fish oil on TNF-α production by PBMC from healthy subjects (Endres et al., N Engl J Med 320:265-71 (1989); Schmidt et al., Scand J Clin Lab Med 56:87-92 (1996); Cooper et al., Clin Nutr 12:321-8 (1993); Blok et al., Eur J Clin Invest 27:1003-8 (1997); Meydani et al., J Clin Invest 92:105-13 (1993); Molvig et al., Scan J Immunol 34:399-410 (1991); Meydani et al., J Nutr 121:547-55 (1991); Caughey et al., Am J Clin Nutr 63:116-22 (1996); Yaqoob et al., Eur J Clin Nutr 30:399-410 (2000); Gallai et al., J Neuroimmunol 56:143-53 (1995) and Kelley et al., Lipids 34:317-24 (1999)), only six report a suppressive effect (Endres et al., N Engl J Med 320:265-71 (1989); Meydani et al., J Clin Invest 92:105-13 (1993); Meydani et al., J Nutr 121:547-55 (1991); Caughey et al., Am J Clin Nutr 63:116-22 (1996); Gallai et al., J Neuroimmunol 56:143-53 (1995) and Kelley et al., Lipids 34:317-24 (1999)).

In healthy males and post-menopausal females, TNF-α production from PBMC shows a remarkable constancy with each individual exhibiting a characteristic level of production of the cytokine (Jacob et al., PNAS 87:1233-7 (1990)). However, among individuals, there is a wide variation in the production of TNF-α. It is postulated that polymorphisms in the promoter regions of the TNF-α and lymphotoxin-α (LT-α, also known as TNF-β) genes influence the amount of TNF-α produced following an inflammatory stimulus (Wilson et al., J Inflamm 45:1-12 (1995)). This appears to have clinical significance. For example, individuals homozygous for the TNF-α −308 (TNF2) allele had 7 times the rate of malaria-related mortality and serious neurological symptoms than individuals who were heterozygous or homozygous for the more common TNF1 allele (Mc Guire et al, Nature 371:508-11 (1994)). Surgical patients developing sepsis who were homozygous for the LT-α +252 (TNFB2) allele had 3.5 times higher mortality rates than individuals homozygous for the TNFB1 allele, and 2.4 times higher than individuals who were heterozygous (Stuber et al., Crit Care Med 24:381-4 (1996)).

Levels of a further cytokine, interleukin-6 (IL-6) have also been postulated as determining an individual's response to fish oil treatment. According to the literature (Fishman et al., J. Clin Invest 102:1369-1376 (1998) and Villuendas et al., J. Clin Endocrinol Metab 87:1134-1141 (2002)) individuals with a base pair of GG at position −174 of the IL-6 gene produce raised levels of IL-6, whereas individuals with a CC base pair at this position produce low levels of IL-6. IL-6 production in the body contributes to the general level of inflammation experienced by individuals. However, the prior art does not link the specific genotypes at position −174 with TNF production. An analysis of the relationship between the genotype at position −174, the level of inflammation and TNF production would enable more accurate targeting of fish oil to those individuals most likely to respond thereto.

All of the studies on the effects of fish oil on TNF-α production reported large standard deviations in TNF-α production, which could suggest a mixture of genotypes in the study population. It is unknown whether polymorphisms in TNF-α, LT-α and IL-6 genes influence the ability of fish oil to suppress TNF-α production. An understanding of these interactions may explain the inconsistencies in the literature and may permit more specific targeting of fish oil treatment for inflammatory disease.

Fish oil is an inexpensive treatment for inflammatory disease. However, rather than suggest to each patient that they should take the supplement “to see whether it helps” which, if applied to every dietary supplement could result in a large financial outlay for the patient with no guarantee of success, it would be preferable to be able to target the fish oil to those patients most likely to benefit from it.

### SUMMARY OF THE INVENTION

The present inventors have recognised that the sensitivity of an individual to the inflammation suppressing effects of fish oil on TNF-α production is linked to genetic variation encoded by, or associated with, the TNF-α −308, LT-α +252 and IL-6 −174 single nucleotide polymorphisms (SNP's). The inflammation suppressing effects of fish oil on TNF-α production have also been shown to be related to the inherent level of production of TNF-α by cells.

Accordingly, the present invention provides, in its first aspect, a method of assessing the susceptibility of an individual to treatment of an inflammatory disease with fish oil, comprising determining the genotype of the individual in relation to polymorphisms at the TNF-α −308, LT-α +252 and/or IL-6 −174 alleles; and inferring therefrom whether said individual responds well to treatment with fish oil.

In its second aspect, the present invention provides a method of assessing the susceptibility of an individual to treatment of an inflammatory disorder with fish oil, comprising:
a) determining the inherent TNF-α status of the individual; and
b) determining the genotype of the individual in relation to polymorphisms at the TNF-α −308, LT-α +252 and/or IL-6 −174 alleles; and
   inferring therefrom whether said individual responds well to treatment with fish oil.

In a further aspect, the present invention provides a method for the treatment of an inflammatory disease in a patient, which comprises assessing the susceptibility of an individual to treatment of an inflammatory disease with fish oil, said assessment comprising:
a) determining the genotype of the individual in relation to polymorphisms at the TNF-α −308, LT-α +252 and IL-6 −174 alleles;
b) inferring therefrom whether said individual responds well to treatment with fish oil; and
   treating said individual with an appropriate amount of fish oil.

In a yet further aspect, the present invention provides a method for the treatment of an inflammatory disease in a patient, which comprises assessing the susceptibility of an individual to treatment of an inflammatory disease with fish oil, said assessment comprising:

a) determining the inherent TNF-α status of the individual;

b) determining the genotype of the individual in relation to polymorphisms at the TNF-α −308, LT-α +252 and IL-6 −174 alleles;

c) inferring therefrom whether said individual responds well to treatment with fish oil; and

treating said individual with an appropriate amount of fish oil.

### DETAILED DESCRIPTION OF THE INVENTION

In its first aspect, the present invention requires the determination of the genotype of the individual in relation to polymorphisms at the TNF-α −308, LT-α +252 and/or IL-6 −174 alleles.

Polymorphisms in the TNF-α (TNF1, TNF2) gene and LT-α (TNFB1, TNFB2) gene will result in three genotypes in each case, homozygous TNF1/1 or TNFB1/1, or homozygous TNF2/2 and TNFB2/2 or heterozygous TNF1/2 and TNFB1/2. In a similar fashion, in respect of the IL-6 gene, individuals will either be homozygous CC or GG or heterozygous CG. Thus, in the present invention an individual may have one of the following genotypes:
homozygous TNF1/1:homozygous TNFB1/1:homozygous IL-6 CC
homozygous TNF1/1:homozygous TNFB1/1:homozygous IL-6 GG
homozygous TNF1/1:homozygous TNFB1/1:heterozygous IL-6 CG
homozygous TNF1/1:homozygous TNFB2/2:homozygous IL-6 CC
homozygous TNF1/1:homozygous TNFB2/2:homozygous IL-6 GG
homozygous TNF1/1:homozygous TNFB2/2:heterozygous IL-6 CG
homozygous TNF1/1:heterozygous TNFB1/2:homozygous IL-6 CC
homozygous TNF1/1:heterozygous TNFB1/2:homozygous IL-6 GG
homozygous TNF1/1:heterozygous TNFB1/2:heterozygous IL-6 CG
homozygous TNF2/2:homozygous TNFB1/1:homozygous IL-6 CC
homozygous TNF2/2:homozygous TNFB1/1:homozygous IL-6 GG
homozygous TNF2/2:homozygous TNFB1/1:heterozygous IL-6 CG
homozygous TNF2/2:homozygous TNFB2/2:homozygous IL-6 CC
homozygous TNF2/2:homozygous TNFB2/2:homozygous IL-6 GG
homozygous TNF2/2:homozygous TNFB2/2:heterozygous IL-6 CG
homozygous TNF2/2:heterozygous TNFB1/2:homozygous IL-6 CC
homozygous TNF2/2:heterozygous TNFB1/2:homozygous IL-6 GG
homozygous TNF2/2:heterozygous TNFB1/2:heterozygous IL-6 CG
heterozygous TNF1/2:homozygous TNFB1/1:homozygous IL-6 CC
heterozygous TNF1/2:homozygous TNFB1/1:homozygous IL-6 GG
heterozygous TNF1/2:homozygous TNFB1/1:heterozygous IL-6 CG
heterozygous TNF1/2:homozygous TNFB2/2:homozygous IL-6 CC
heterozygous TNF1/2:homozygous TNFB2/2:homozygous IL-6 GG
heterozygous TNF1/2:homozygous TNFB2/2:heterozygous IL-6 CG
heterozygous TNF1/2:heterozygous TNFB1/2:homozygous IL-6 CC
heterozygous TNF1/2:heterozygous TNFB1/2:homozygous IL-6 GG; or
heterozygous TNF1/2:heterozygous TNFB1/2:heterozygous IL-6 CG.

Although on paper the above are all of the possible gene combinations, in reality, not all of the combinations will occur. Certain combinations may be prevented from forming, for example due to the linkage of some genes, causing linkage disequilibrium. In addition to this, the gene TNF2/2 is very rare, occurring in less than 10% of the population.

In a preferred embodiment of the present invention, it is preferred that the genotype at the LT-α gene is heterozygous TNFB1/2.

In a further preferred embodiment of the present invention, it is preferred that the genotype at the IL-6 gene is homozygous GG.

In a more preferred embodiment of the present invention, it is preferred that the genotype at the LT-α and IL-6 genes is heterozygous TNFB1/2 and IL-6 GG respectively.

In a preferred embodiment of this aspect of the present invention, the genotype at one of the TNF-α, LT-α and IL-6 alleles is determined. It is more preferred however, that the genotype of two or more of the TNF-α, LT-α and IL-6 alleles is determined and most preferred that the genotype of both the LT-α and IL-6 alleles is determined.

In its second aspect, the present invention requires the determination of the inherent TNF-α status of an individual.

The inherent TNF-α status of an individual is a measurement of the ability of that individual's white blood cells to make TNF. The inherent TNF-α status of an individual in a disease-free or substantially disease-free state demonstrates a remarkable constancy. The production of TNF-α is generally not affected by age or sex of the individual. By disease-free or substantially disease-free is meant that the individual does not suffer from any type or significant level of inflammatory disorder. The inherent TNF-α status of an individual is thus preferably determined when the individual is not suffering from any type or significant level of inflammatory disorder.

Determination of the inherent TNF-α status of an individual may be made using procedures known to the person skilled in the art. Typically samples of whole blood are collected and peripheral blood mononuclear cells (PBMC) isolated therefrom. Techniques for isolation of PBMC are known and include, for example, treatment of whole blood with lithium heparin, followed by centrifugation to isolate PBMC, after which TNF-α concentrations can be measured, for example, using standard methods such as EASIA® ELISA kits (Biosource International, Nivelles, Belgium).

TNF-α producers can be divided into numerous groups depending on the concentration of TNF-α produced by their PBMC and thereby defining their inherent TNF-α status. It is generally preferred for the purposes of the present invention that producers be classified into three groups, for example as high, medium or low producers. Although the exact amounts of TNF-α produced by the PBMC are not essential to define the above three groups, it is generally preferred that a high producer has a concentration of TNF-α of about 850-2500 ng/L of incubate from 1×10⁹ cells, a medium producer has a TNF-α concentration of 2500-5000 ng/L of incubate from 1×10⁹ cells and a high producer has a concentration of TNF-α of about 5000-14000 ng/L of incubate from 1×10⁹ cells.

Table 1, below, illustrates the effects of fish oil on TNF-α production.

TABLE 1

Conc. TNF-α - ng/L

| | No. | Pre- | Post- |
|---|---|---|---|
| Tertile | subjects | Supplementation | supplementation |
| | 111 | 4821 ± 4177 | 4643 ± 3338 |
| Low | 37 | 1458 ± 600 | 3809 ± 2571* |
| Medium | 37 | 3728 ± 936 | 4796 ± 3270 |
| High | 37 | 9277 ± 4338 | 5323 ± 3941* |

*significantly different from pre-supplementation value (P < 0.05; Student's paired t-test)

Differences between pre- and post-fish oil supplementation values for TNF-t production were determined using Student's paired t-test. It is apparent from the above results that sensitivity to fish oil administration is influenced by pre-supplementation or inherent TNF-α production. In the highest tertile, mean TNF-α production was reduced by 43%. TNF-α production was reduced in the middle tertile, although not by a significant amount and in the lowest tertile, TNF-α production was increased by 160%.

In view of the above results, the present inventors have determined that an individual whose inherent TNF-α production causes them to fall into the “low producer” category, i.e. an individual having from about 850-2500 ng TNF-α/L of incubate from 1×10⁹ cells, is not likely to respond well to fish oil treatment. In contrast, an individual whose inherent TNF-α production causes them to fall into the “high producer” category, i.e. an individual having from about 5000-14000 ng TNF-α/L of incubate from 1×10⁹ cells is likely to respond well to fish oil treatment.

A genomic sample suitable for use in such a method may be isolated from any suitable client or patient cell sample. For convenience, it is preferred that the DNA is isolated from cheek (buccal) cells. This enables easy and painless collection of cells.

Cells may be isolated from the inside of the mouth using a disposable scraping device with a plastic or paper matrix “brush”, for example, the C.E.P. Swab™ (Life Technologies Ltd., UK). Cells are deposited onto the matrix upon gentle abrasion of the inner cheek, resulting in the collection of approximately 2000 cells (Aron, Y. et al (1994) Allergy 49 (9): 788-90). The paper brush can then be left to dry completely, ejected from the handle placed into a micro-centrifuge tube for storage prior to analysis.

Genomic DNA from the cell samples may be isolated using conventional procedures. For example DNA may be immobilised onto filters, column matrices, or magnetic beads. Numerous commercial kits, such as the Qiagen QIAamp kit (Qiagen, Crawley, UK) may be used. Briefly, the cell sample may be placed in a microcentrifuge tube and combined with Proteinase K, mixed, and allowed to incubate to lyse the cells. Ethanol is then added and the lysate is transferred to a QIAamp spin column from which DNA is eluted after several washings.

The amount of DNA isolated by the particular method used may be quantified to ensure that sufficient DNA is available for the assay and to determine the dilution required to achieve the desired concentration of DNA for PCR amplification. For example, the desired target DNA concentration may be in the range 50 ng and 150 ng. DNA concentrations outside this range may impact the PCR amplification of the individual alleles and thus impact the sensitivity and selectivity of the polymorphism determination step.

The quantity of DNA obtained from a sample may be determined using any suitable technique. Such techniques are well known to persons skilled in the art and include UV (Maniatis T., Fritsch E. F., and Sambrook J., (1982) Molecular Cloning A Laboratory Manual, Cold Spring Harbor Laboratory, Cold Springs Harbor, N.Y.) or fluorescence based methods. As UV methods may suffer from the interfering absorbance caused by contaminating molecules such as nucleotides, RNA, EDTA and phenol and the dynamic range and sensitivity of this technique is not as great as that of fluorescent methods, fluorescence methods are preferred. Commercially available fluorescence based kits such as the PicoGreen dsDNA Quantification (Molecular Probes, Eugene, Oreg., USA).

Prior to the testing of a sample, the nucleic acids in the sample may be selectively amplified, for example using Polymerase Chain Reaction (PCR) amplification, as described in U.S. Pat. Nos. 4,683,202 and 4,683,195.

Preferred primers for use in the present invention are from 18 to 23 nucleotides in length, without internal homology o primer-primer homology.

Furthermore, to ensure amplification of the region of interest and specificity, ARMS-PCR or allele specific PCR methodology is used. In this way, in each PCR reaction, the 3′ end of one primer is located exactly at the site of the SNP, so that amplification can only occur if the nucleotide base corresponding to one particular allele is present. Tow PCR reactions are performed per SNP, one specific for each allele. One primer is common to both PCR reactions, while separate allele specific primers are added for each reaction, e.g. the common primer could be “forward” with allele 1 (reaction 1) or allele 2 (reaction 2) added as “reverse” primers, or vice-versa.

This approach is based upon the original discovery that specificity in a PCR reaction is dependent upon precise matching of the terminal 3′ end of a PCR primer and its target DNA sequence. Using this approach, point mutations may be distinguished from wild-type sequences using a single generic primer combined with one of two antisense primers in separate PCR reactions. One of the antisense primers is precisely matched with the wild-type sequence at its 3′ end, while the second primer is precisely matched with the mutant sequence at its 3′ end. Therefore one PCR reaction will only amplify the wild-type sequence and the other will only amplify the mutant sequence. This method, originally termed the ‘amplification refractory mutation system-PCR’ (ARMS-PCR) (Newton et al 1989), is dependent upon the fact that Taq DNA polymerase lacks 3′ to 5′ exonucleolytic proof-reading activity, so that Watson-Crick mismatches at the 3′ end of the primer-template duplex cannot be corrected, which would result in mispriming. Successful application of the ARMS-PCR approach also requires stringent conditions for primer annealing in the PCR reaction. A vital component of this methodology is inclusion of a second primer pair which amplifies a sequence from a second gene, to act as an in-tube positive control for successful or failed PCR amplification in that tube.

Preferred examples of primer pairs which may be used for analysing the TNF-α, LT-α and IL-6 genes are shown in Table 2, together with the control primers which amplify a sequence from the third intron of the human leukocyte antigen DRB1, to act as an internal control for successful PCR.

TABLE 2

| | | Primer Sequence | Seq | Prod. Size |
|---|---|---|---|---|
| SNP | Primer Name | (5′-3′) | ID | (BPs) |

| | | | | |
|---|---|---|---|---|
| TNF-α | TNF308 common | TCTCGGTTTCTTCTCCATCG | 1 | 184 |
| -308 | TNF308G | ATAGGTTTTGAGGGGCATGG | 2 | |
| | TNF308A | ATAGGTTTTGAGGGGCATGA | 3 | |
| LT-a +252 | LT252 common | AGATCGACAGAGAAGGGGACA | 4 | 94 |
| | LT252G | CATTCTCTGTTTCTGCCATGG | 5 | |
| | LT252A | CATTCTCTGTTTCTGCCATGA | 6 | |
| IL-6 −174 | IL6174 common | TTTGTTGGAGGGTGAGGGTGG | 7 | 108 |
| | IL6174G | TTCCCCCTAGTTGTGTCTTGCG | 8 | |
| | IL6174C | TTCCCCCTAGTTGTGTCTTGCC | 9 | |
| Control | 63 | TGCCAAGTGGAGCACCCAA | 10 | 796 |
| primers | 64 | GCATCTTGCTCTGTGCAGAT | 11 | |

Having obtained a sample of DNA, preferably with amplified regions of interest, the individual polymorphisms may be identified. Identification of the markers for the polymorphisms involves the discriminative detection of allelic forms of the TNF-α, LT-α and IL-6 genes that differ by nucleotide substitution at positions −308, +252 and/or −174 respectively.

Methods for determining the presence of known nucleotide differences are well known to the skilled person. These may include, but are not limited to:
Hybridization with allele-specific oligonucleotides (ASO), (Wallace, R. B. et al (1981) Nucleic Acids Research. 9:879-894; Ikuta, S. et al (1987) Nucleic Acids Research. 15:797-811; Nickerson, D. et al (1990) PNAS USA 87:8923-8927, Verlaande Vries, M et al (1986) Gene. 50:313-320, Saiki, R. K. et al (1989) PNAS. USA 86:6230-6234 and Zhang, Y. et al (1991) Nucleic Acids Research. 19: 3929-3933)
Allele specific PCR, (Newton, C. R. et al (1989). Nucleic Acids Research. 17:2503-2516, Gibbs, R. A. et al (1989) Nucleic Acids Research. 17:2437-2448).

The following reference gives full details of the TNF alpha/LT alpha genotyping system used:
Howell W M, Bateman A C, Turner S J, Theaker J M (2002). Influence of TNFα and LTα single nucleotide polymorphisms on susceptibility to and prognosis in cutaneous malignant melanoma in the British population European Journal of Immunogenetics, 29, 17-23.

A few of other examples describing details of cytokine genotyping by ARMS-PCR include:
McCarron S L, Edwards S, Evans P R, Gibbs R, Dearnaley D P, Dowe A, Southgate C, The CRC/BPG UK Familial Prostate Cancer Study Collaborators, Easton D F, Eeles R A, Howell W M (2002) Influence of cytokine gene polymorphisms on the development of prostate cancer. Cancer Research, 62, 3369-3372.
Howell W M, Turner S J, Bateman A C, Theaker J M (2001). IL-10 promoter polymorphisms influence tumour development in cutaneous malignant melanoma. Genes and Immunity, 2, 25-31.
Poole K L, Gibbs P J, Evans P R, Sadek S A, Howell W M (2001) Influence of patient and donor cytokine genotypes on renal allograft rejection: evidence from a single study. Transplant Immunology, 8, 259-265.

Further references of interest include:
Solid-phase minisequencing (Syvanen, A. C. et al (1993) Am. J. Human Genet. 52:46-59).
Oligonucleotide ligation assay (OLA) (Wu, D. Y., et al (1989) Genomics. 4:560-569, Barany, F. (1991) PNAS USA 88:189-193, Abravaya, K. et al 1995. Nucleic Acids Research. 23:675-682).
The 5′ fluorogenic nuclease assay (Lee, E. et al J. Toxicol. Soc. 23: 140-142, (1998), U.S. Pat. No. 4,683,202, U.S. Pat. No. 4,683,195, U.S. Pat. No. 5,723,591 and U.S. Pat. No. 5,801,155).
Restriction fragment length polymorphism (RFLP), (Donis-Keller H. et. al. (1987) Cell, 51, 319-337).

In a preferred embodiment, the loci for the three genes may be assessed via a specialised type of PCR used to detect polymorphisms, commonly referred to as the Taqman® assay and performed using a AB77000R 7900HT instruments (Applied Biosystems, Warrington, UK). In this method, a probe is synthesised which hybridises to a region of interest containing the polymorphism. The probe contains three modifications: a fluorescent reporter molecule, a fluorescent quencher molecule and a minor groove binding chemical to enhance binding to the genomic DNA strand. The probe may be bound to either strand of DNA. For example, in the case of binding to the coding strand, when the Taq polymerase enzyme begins to synthesise DNA from the 5′ upstream primer, the polymerase will encounter the probe and begin to remove bases from the probe one at a time using a 5′-3′ exonuclease activity. When the base bound to the fluorescent reporter molecule is removed, the fluorescent molecule is no longer quenched by the quencher molecule and the molecule will begin to fluoresce. This type of reaction can only take place if the probe has hybridised perfectly to the matched genomic sequence. As successive cycles of amplification take place, i.e. more probes and primers are bound to the DNA present in the reaction mixture, the amount of fluorescence will increase and a positive result will be detected. If the genomic DNA does not have a sequence that matches the probe perfectly, no fluorescent signal is detected.

The results of the genetic polymorphism analysis may be used in combination with the results of the determination of the producer level with respect to inherent TNF-α production to allow a determination to be made of the susceptibility of the individual to fish oil treatment. Table 3, below, demonstrates the distribution of TNF-α, LT-α and IL-6 genotypes in the study population in relation to inherent TNF-α producer status.

TABLE 3

| | TNF-α | | | LT-α | | | | | |
|---|---|---|---|---|---|---|---|---|---|
| | genotype | | | Genotype (B) | | | IL-6 genotype | | |
| Tertile | 1/1 | 1/2 | 2/2 | 1/1 | 1/2 | 2/2 | GG | GC | CC |
| All | 76 | 33 | 2 | 21 | 59 | 31 | 49 | 45 | 17 |
| Low | 25 | 11 | 1 | 8 | 23 | 6 | 16 | 11 | 10 |
| Medium | 25 | 11 | 1 | 8 | 19 | 10 | 14 | 18 | 5 |
| High | 26 | 11 | 0 | 5 | 17 | 15* | 19 | 16 | 2 |

*indicates distribution of LT-α and IL-6 genotypes significantly different from lowest tertile of TNF-α production (P < 0.001: χ² test).

As may be seen from the above table, the percentage of subjects falling into the TNF1/1, TNF1/2 and TNF2/2 genotypes was approximately 68%, 30% and 2% respectively. The percentage of subjects falling into the TNFB1/1, TNFB1/2 and TNFB2/2 genotypes was 19%, 53% and 28% respectively and the percentage of subjects falling into the IL-6GG, IL-6GC and IL-6CC genotypes was 53%, 56% and 29% respectively. TNF-α genotype appeared to be unrelated to TNF-α production since the distribution of TNF1 and TNF2 alleles was almost identical for the subjects in all tertiles of pre-supplementation TNF-α production. The frequency of the TNFB2/B2 and IL-6 GG genotypes was related positively to TNF-α production, increasing in the case of TNFB2/2 from 19% in the lowest tertile to 48% in the highest tertile and in the case of IL-6 showing a smaller increase of from 33% in the lowest tertile to 39% in the highest. The frequency of the TNFB1/B2 genotype and the IL-6 CC genotype declined as inherent TNF-α production increased.

The influence of genotype on the ability of fish oil to reduce TNF-α production from LPS stimulated peripheral blood mononuclear cells is also demonstrated in Table 4. It is apparent from this table that the presence of either the IL-6 GG or TNFB 1/2 polymorhisms alone causes the individual with either of those genotypes to be more susceptible to the beneficial lowering of TNF-α production following fish oil administration. Furthermore, a combination of the TNFB1/2 and IL-6GG genotypes is most likely to result in enhancing the effects of fish oils reduction of TNF production, as is shown by the fact that 56% of subjects with that genotype show a reduction in TNF-α production following fish oil administration (Table 4).

TABLE 4
Influence of genotype on the ability of fish oil (6 g/d
for 12 weeks) to reduce TNF-alpha production from LPS stimulated
peripheral blood mononuclear cells of men.

| | | | Number | Percentage |
|---|---|---|---|---|
| | | Number | showing | of subjects |
| | | showing | no change or | with genotype |
| | | a fall in | a rise in | showing a |
| Genotype | Total | TNF after | TNF after | fall in TNF |
| combinations | subjects | fish oil | fish oil | after fish oil |
| TNFB11 & | 12 | 4 | 8 | 33 |
| IL-6 GG | | | | |
| TNFB12 & | 45 | 25 | 20 | 56 |
| IL-6 GG | | | | |
| TNFB22 & | 32 | 15 | 16 | 47 |
| IL6 GG | | | | |
| TNFB11 & | 12 | 4 | 8 | 33 |
| IL-6 GC | | | | |
| TNFB12 & | 41 | 19 | 22 | 46 |
| IL-6 GC | | | | |
| TNFB22 & | 34 | 15 | 19 | 44 |
| IL-6 GC | | | | |
| TNFB11 & | 5 | 1 | 4 | 20 |
| IL-6 CC | | | | |
| TNFB12 & | 18 | 6 | 12 | 33 |
| IL-6 CC | | | | |
| TNFB22 & | 9 | 1 | 8 | 11 |
| IL-6 CC | | | | |
| TNFB11 | 29 | 9 | 20 | 31 |
| TNFB12 | 104 | 51 | 53 | 49 |
| TNFB22 | 75 | 31 | 44 | 41 |
| IL-6 GG | 89 | 44 | 45 | 49 |
| IL-6 GC | 87 | 38 | 49 | 44 |
| IL-6 CC | 32 | 8 | 24 | 25 |
| Irrespective | 208 | 90 | 118 | 43 |
| of genotype | | | | |

A subject is deemed to have shown a fall ion TNF-alpha production after fish oil if post-fish oil value is >10% lower than pre-fish oil value.
TNF-beta +252 and IL-6 −174 Single nucleotide polymorphisms characterized.
10-10-2002

For the purposes of the present invention, fish oil is oil extracted from the flesh and organs of fish which contains at least 28% n-3 PUFAs, of which about 60% is eicosapentaenoic acid and about 40% is docosahexaenoic acid. The fish oil may be extracted from any oily fish source. Particularly appropriate in this respect are mackerel, sprats, herring, tuna and wild salmon, these fish being rich sources of n-3 PUFAs. The remaining components of the fish oil are typically a mixture of saturated and monounsaturated fatty acids which do not appear to have any bearing on the activity of the fish oil.

A suitable inflammatory disorder may include any disorder in which a reduction in inflammation is desirable, including an inflammatory skin disorder such as atopic dermatitis, contact dermatitis, eczema, psoriasis and other inflammatory disorders such as Perianal Crohn's disease and arthritis, for example, rheumatoid or psoriatic arthritis. Fish oil treatment has been found to be of particular benefit in the treatment of rheumatoid arthritis.

In some embodiments, a method may comprise the further step of administering fish oil to the individual. Fish oil may be administered alone or in combination with one or more of vitamin B12/B6 and antioxidants, for example vitamin C, vitamin E, lycopene, beta-carotene and minerals such as magnesium, manganese, selenium and zinc.

Administration may be in the form of a medicament such as a tablet or pill, which, for example comprises the active ingredient and a suitable excipient, or in the form of a foodstuff rich in fish oil. Suitable foodstuffs would include oily fish such as mackerel, sprats, herring, tuna and wild salmon.

A method may comprise the further step of providing a dietary regime for said individual comprising foodstuffs comprising elevated levels of one or more of folic acid, vitamin B6/B12 and vitamin C.

Another aspect of the invention provides the use of composition comprising fish oil in the manufacture of a medicament for use in the treatment of an inflammatory disorder in an individual who is polymorphic for one or more of the TNF-α −308, LT-α +252 and IL-6 −174 polymorphisms.

The term “treatment” as used herein in the context of treating a condition, pertains generally to treatment and therapy, whether of a human or an animal (e.g. in veterinary applications), in which some desired therapeutic effect is achieved, for example, the inhibition of the progress of the inflammatory condition, and includes a reduction in the rate of progress, a halt in the rate of progress, amelioration of the inflammatory condition, and cure of the inflammatory condition. Treatment as a prophylactic measure (i.e. prophylaxis) is also included.

The term “therapeutically-effective amount” as used herein, pertains to that amount of an active compound, or a material, composition or dosage from comprising an active compound, which is effective for producing some desired therapeutic effect, commensurate with a reasonable benefit/risk ratio.

Fish oil or a pharmaceutical composition comprising fish oil, may be administered to a subject by any convenient route of administration, including but not limited to oral administration (e.g. by ingestion) or parenteral administration, e.g. by subcutaneous, intramuscular or intravenous injection.

The subject may be a eukaryote, an animal, a vertebrate animal, a mammal, a rodent, murine, canine, feline, equine bovine, ovine or human.

While it is possible for the fish oil to be administered alone, it is preferable to present it as a pharmaceutical composition (e.g. formulation) comprising at least the fish oil together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing fish oil together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, oils, suppositories, boluses or sustained release formulations.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

A more recently devised approach for parenteral administration employs the implantation of a slow-release or sustained-release system, such that a constant level of dosage is maintained. See, e.g., U.S. Pat. No. 3,710,795.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of fish oil is in the range of about 4 g to about 8 g per day, more preferably about 6 g per day. This may be in the form of a single bolus dose or more preferably in multiple applications or a sustained release preparation. Factors such as age, weight, sex and presence or absence of other, non-inflammatory, diseases, will generally not have a bearing on the suitable daily dose of fish oil.

The precise format for performing methods of the invention may be varied by those of skill in the art using routine skill and knowledge.

Of course, the person skilled in the art will design any appropriate control experiments with which to compare results obtained in methods of the invention.

Aspects of the present invention will now be illustrated with reference to the experiments and results below, by way of example and not limitation. Further aspects and embodiments will be apparent to those of ordinary skill in the art.

All documents mentioned in this specification are hereby incorporated herein by reference.

### EXPERIMENTS

1. Subjects and Study Design

Healthy male subjects (n=111), age 28±8 years (range 20-57 years), body weight 77±11 kg (range 50-103 kg) and body mass index 24±4 kg/m² (range 18-34 kg/m²), were recruited from the Southampton area. Smokers and individuals with inflammatory disease or on anti-inflammatory drugs were excluded from the study. Subjects continued with their normal lifestyles and diet but, in addition, consumed 6 g/d of encapsulated fish oil (providing 1.8 ng n-3 PUFAs/d) (Maxepa, Seven Seas Ltd, Hull, UK) for 12 weeks. Prior to providing blood subjects fasted overnight, for at least 12 hours. Three separate blood samples were taken sequentially at the start and on completion of the period of fish oil supplementation. First 20 ml of blood was taken into a vacutainer tube containing lithium heparin; this blood was used for preparation of PBMC. Then 5 ml of blood was taken into a coagulant-free vacutainer tube: this blood was used for preparation of serum to measure C-reactive protein (CRP) concentrations. Finally 5 ml of blood was taken into a vacutainer containing EDTA: this blood was used to prepare DNA for genotyping.

Serum CRP concentrations were measured in order to detect the presence of infection or inflammation in the subjects at the time of blood sampling. Individuals with CRP concentrations>100 mg/L from either blood sample were excluded from the study.

2. TNF-α Induction and Measurement

PBMC were isolated by centrifugation of heparinised blood on Histpaque-1077 (Sigma Chemical Co., Poole, UK) (Yaqoob et al,. Eur J Clin Nutr 30:399-410 (2000)) and were resuspended in RPMI culture medium containing 2 mmol/L glutamine and 50 ml/l autologous plasma. PBMC (2×10⁶) were cultured in 24 well tissue culture plates in the presence of a final concentration of 15 mg/l Eschericia coli 0111:B4 endotoxin (Sigma Chemical Co., Poole, UK) in a final culture volume of 2 ml. After 24 h at 37° C. in a 5% CO₂/95% air atmosphere, the culture plates were centrifuged and the supernatants frozen at −80° C. until analysis. TNF-α concentrations were measured using EASIA® ELISA kits (Biosource International, Nivelles, Belgium). The inter- and intra-assay coefficients of variation were <10% and the limit of detection was 3 ng/l.

3. Genotyping for TNF-α and LT-α Alleles

An aliquot of blood which had been collected into EDTA was genotyped for the TNF-α −308 (TNF1, TNF2) and LT-α +252 (TNFB1, TNFB2) single nucleotide polymorphisms (SNP's). These SNPs were selected due to their documented, but variable, association with TNF-α production (Kroeger et al., Cytokine 12:110-119 (2000) and Warzocha et al., Blood 91:3564-81 (1998)). Genomic DNA was extracted by a salting out procedure (Miller et al., Nucleic Acid Res 16:1215 (1988)). Each SNP was detected using a two reaction amplification refractory mutation system polymerase chain reaction (ARMS-PCR) approach based on previously published methods (Howell W M et al., European Journal of Immunogenetics, 29:17-23 (2002); Perrey et al., Transpl Immunol 7:127-8 (1999)). In this approach, two separate PCR reactions per SNP are performed. Each PCR reaction also contained an additional pair of PCR primers, amplifying a sequence from the third intron of the human leucocyte antigen DRB1 gene to act as an internal control for successful PCR. All PCR reactions were performed in 10 μl reaction volumes and final reagent concentrations were as follows: 1×AS reaction buffer (Abgene, Epsom, UK), 200 μmol/l each dNTP, 120 g/l sucrose, 200 μmol/l cresol red, 1 μmol/l each specific or common primer, 0.2 μmol/l each internal control primer, 0.25 units Thermoprime^{PLUS} DNA polymerase (Abgene, Epsom, UK), 1.75 mmol/l MgCl₂ and 25-100 ng DNA. PCR primer sequences and product sizes for each SNP amplicon are given in Table 2. PCR reaction conditions were performed using a Primus 96 Plus thermal cycler (MWG Biotech, Germany) according to the following cycling conditions: 96° for 60 s, followed by ten cycles of 96° for 15 s, 65° for 50 s, 72° for 40 s; then twenty cycles of 96° for 190 s, 60° for 50 s, 72° for 40 s. PCR products were loaded directly onto 2% agarose gels containing 0.5 g/l ethidium bromide, electrophoresed and visualised by photography under UV transillumination.

4. Plasma Phospholipid Fatty Acid Composition

Compliance to the dietary fish oil treatment was assessed by determination of the fatty acid composition of plasma phospholipids. Total lipid was extracted from plasma with chloroform/methanol (2:1 v/v) and phospholipids were isolated by thin layer chromatography using a mixture of hexane/diethyl ether/acetic acid (90:30:1 v/v/v) as the elution phase. Fatty acid methyl esters were prepared by incubation with 10 g/l boron trifluoride in methanol at 8 ° C. for 60 mins. Fatty acid methyl esters were isolated by solvent extraction, dried and separated by gas chromatography in a Hewlett-Packard 6890 gas chromatograph (Hewlett Packard, Avondale, Pa.) fitted with a 30 m×0.32 mm BPX70 capillary column, film thickness 0.25 μm. Helium at 1.0 ml/min was used as the carrier gas and the split/splitless injector was used with a split/splitless ratio of 20:1. Injector and detector temperatures were 275° C. The column oven temperature was maintained at 170° C. for 12 min after sample injection and was programmed then to increase from 170 to 210° C. at 5° C./min before being maintained at 210° C. for 15 min. The separation was recorded with HP GC Chem Station software (Hewlett Packard, Avondale, Pa.). Fatty acid methyl esters were identified by comparison with standards run previously.

5. Statistical Analysis

Unless otherwise indicated values are expressed as mean±SD. Differences in the distribution of TNF-α and LT-α genotypes among the tertiles of TNF-α production before fish oil supplementation were examined using the χ² test. Differences between the pre- and post-fish oil supplementation values for TNF-α production and for the proportions of various fatty acids in plasma phospholipids were determined using Students paired t-test. Differences in TNF-α production among different genotypes, either before or after fish oil supplementation, were determined by one-factor ANOVA. The influence of genotype, of tertile of pre-supplementation TNF-α production and of their interaction on the effect of fish oil on TNF-α production were determined by two-factor ANOVA. In all cases, the level of significance was set at 0.05 and Bonferonni's correction for multiple comparisons was used. All statistical comparisons were made using SPSS Version 10 (SPSS Inc, Chicago, Ill.).

Results

1. Plasma Phospholipid Fatty Acid Composition

All subjects showed an increase in the proportions of eicosapentaenoic acid and docosahexaenoic acid in their plasma phospholipids, with mean increases of 370 and 94 respectively, at the end of the supplementation period (Table 5). Increased appearance of the fish oil-derived n-3 PUFAs was accompanied by a significant decrease in the proportion of arachidonic acid in plasma phospholipids.

TABLE 5

| | % by wt of total fatty acids | |
|---|---|---|
| Fatty Acid | Pre-supplementation | Post-supplementation |
| Eicosapenaenoic acid | 0.72 ± 0.09 | 3.34 ± 0.29* |
| (20:5n − 3) | | |
| Docosahexaenoic acid | 1.97 ± 0.27 | 3.68 ± 0.27* |
| (22:6n − 3) | | |
| Arachidonic acid | 7.66 ± 0.69 | 5.23 ± 0.79* |
| (20:4n − 6) | | |

*indicates significantly different from pre-supplementation value (P < 0.01; Student's paired t-test)

The distribution of genotypes within the study population and relationship with TNF-α production and the influence of fish oil on TNF-α production are discussed above.

2. Influence of TNF-α, LT-α and IL-6 Genotype on Response to Fish Oil

The suppressive effect of fish oil among high TNF-α producers occurred irrespective of TNF-α, LT-α or IL-6 genotype (Table 6). However, there was a significant interaction between TNF-α genotype and inherent TNF-α production in determining the extent of the decline in TNF-α production which followed fish oil supplementation (P for interaction=0.035; two-factor ANOVA).

TABLE 6

| Tertile of inherent | | TNF-α genotype | | LT- α genotype | | |
|---|---|---|---|---|---|---|
| TNF - α production | | 1/1 | 1/2 | B1/B1 | B1/B2 | B2/B2 |
| Lowest | Before FO | 1479 ± 602 | 1294 ± 713 | 1132 ± 556 | 1562 ± 592 | 1187 ± 757 |
| | Change | 2483 ± 2543^{a} | 2365 ± 3040^{a} | 2704 ± 1345 | 2088 ± 2972 | 3442 ± 2602 |
| Middle | Before FO | 3655 ± 962 | 3883 ± 953 | 3910 ± 1066 | 3544 ± 964 | 3884 ± 790 |
| | Change | 658 ± 3066^{b} | 1238 ± 3365^{a} | 2040 ± 2558 | 203 ± 3398 | 1049 ± 2680 |
| Highest | Before FO | 8653 ± 3126 | 10748 ± 6127 | 11570 ± 4391 | 7553 ± 1791 | 10464 ± 5507 |
| | Change | −2923 ± 4429^{c} | −6388 ± 8297*^{b} | −7161 ± 7426 | −3475 ± 2954 | −3246 ± 7800 |

Further analysis showed that the decline in TNF-α production among individuals in the highest tertile of pre-supplementation TNF-α production was significantly greater (P=0.02) if they possessed the TNF1/2 genotype than if they possessed the TNF1/1 genotype (Table 6). The interaction between LT-α genotype and inherent TNF-α production in determining the extent of the decline in TNF-α production which followed fish oil supplementation failed to reach statistical significance (P for interaction=0.062; two-factor ANOVA). Fish oil was able to suppress production of TNF-α by cells from some individuals among the low and medium tertiles of inherent TNF-α production. The TNFB1/B2 allele appeared to be important in determining sensitivity to fish oil among these individuals. Thus, all 8 subjects in the lowest tertile of inherent TNF-α production who responded to fish oil treatment with a reduction in TNF-α production had the TNFB1/B2 genotype. In the middle tertile of inherent TNF-α production, 12 out of 16 subjects who responded in this way had the TNFB1/B2 genotype. In the highest tertile of inherent TNF-α production, the TNFB1/B2 genotype only characterised half of the subjects (16 out of 32) who responded to fish oil with a reduction in TNF-α production.

Discussion

Our data suggest that the sensitivity of an individual to the suppressive effects of n-3 PUFAs on TNF-α production is linked to the inherent level of production of the cytokine by cells from the individual prior to supplementation and to genetic variation encoded by, or associated with, the TNF-α −308, LT-α +252 and IL-6 −174. SNPs. Paradoxically fish oil appears to enhance TNF-α production in some subjects, particularly those in the lowest tertile of pre-supplementation production. The ability of fish oil to enhance rather than reduce TNF-α production is not unexpected. During inflammation phospholipase A₂ hydrolyses membrane phospholipids, thus making arachidonic acid available for the production of the pro-inflammatory eicosanoids prostaglandin E₂ (PGE₂) and leukotriene B₄ (LTB₄). In vitro studies have shown that PGE₂ and LTB₄ have opposing effects on pro-inflammatory cytokine production, the former having an inhibitory and the latter a stimulatory influence (Endres et al., N Eng J Med 320:265-71 (1989) and Choi et al., Cell Immunol 170:178-84 (1996)). Fish oil may alter pro-arachidonic acid in the cell membrane. Such an effect would decrease PGE₂ and LTB₄ production and increase the formation of PGE₃ and LTB₅. These eicosanoids have lower bioactivity than PGE₂ and LTB₄. Thus, the overall effect on TNF-α production (inhibition or stimulation) will depend upon the balance among the different stimulatory and inhibitory eicosanoids produced from arachidonic acid and eicosapentaenoic acid.

As outlined earlier, genetic influences are important in influencing TNF-α production. The frequencies of the TNF1, TNF2, TNFB1, TNFB2, IL-6GG and IL-6CC alleles in the present study accord closely with published values from studies of healthy British and other European subjects (Perrey et al., Transpl Immunol 6:193-7 (1998), Fanning et al., Tissue Antigens 50:23-31 (1997) and Brinkman et al., Br J Rheumatol 36:516-21 (1997)) and are in close agreement with those derived from independent studies in our laboratory (Howell et al., Eur J Immunogenet 29:17-23 (2002)). Thus, the group of subjects studied here is representative of the population from which it is drawn, at least with respect to the frequencies of TNF-α, LT-α and IL-6 genotypes examined. The observed positive association between TNFB2 homozygosity and inherent TNF-α production confirms the findings of Stuber et al., Crit Care Med 24:381-4 (1996) and Pociot et al., Eur J Immunol 23:224-31 (1993). However, we do not confirm an association between TNF-α −308 genotype and TNF-α production. When the genetic characteristics of individuals in the three tertiles of inherent TNF-α production are examined in relation to the ability of fish oil to reduce TNF-α production, a complex interaction was apparent. The results of the present investigation suggest firstly that most (in this case 86%) individuals with a high inherent level of TNF-α production are sensitive to the anti-inflammatory effects of fish oil, secondly that medium and high inherent TNF-α production is associated with homozygosity for the TFNB2 allele, thirdly that individuals with medium or low levels of inherent TNF-α production are more likely to experience the anti-inflammatory effects of fish oil if they are heterozygous for the TNFB alleles and fourthly that possession of the IL-6 −174 CC genotype is associated with a lower level of responsiveness to the anti-inflammatory effects (with regard to TNF production) of fish oil. It is also the case that individuals who are TNFB2/B2 are less likely to experience the anti-inflammatory effects of fish oil, independent of their level of inherent TNF-α production.

The present study is the largest investigation into the effects of dietary fish oil supplementation on ex vivo TNF-α production from human PBMC currently reported in the scientific literature (Calder et al., Nutr Res 21:309-41 (2001)). The data from this study when aggregated without consideration of each subjects inherent ex vivo TNF-α production, or TNF-α, LT-α or IL-6 genotype, agree with other studies which suggest that fish oil does not exert a modulatory effect on such production (Schmidt et al., Scand J Clin Lab Med 56:87-92 (1996); Cooper at al., Clin Nutr 12:321-8 (1993); Blok et al., Eur J Clin Invest 27:1003-8 (1997); Molvig et al., Scand J Immunol 34:399-410 (1991) and Yaqoob et al., Eur J Clin Nutr 30:399-410 (2000)). A wide range of doses of fish oil have been employed in similar studies (0.55 to 6 g n-3 PUFAs/d). Suppressive effects of fish oil on TNF-α production have generally been demonstrated in studies that have employed doses of n-3 PUFAs greater than that employed in the present study (Endres et al., N Engl J Med 320:265-71 (1989); Gallai et al., J Neuroimmunol 56:143-53 (1995) and Kelley et al., Lipids 34:317-24 (1999)). This is not, however, universally the case as some studies using higher doses have shown no effect on TNF-α production (Blok et al., Eur J Clin Invest 27:1003-8 (1997); Molvig et al., Scand J Immunol 34:399-410 (1991) and Yaqoob et al., Eur J Clin Nutr 30:399-410 (2000)). Of the five studies (Schmidt et al., Scand J Clin Lab Med 56:87-92 (1996); Cooper at al., Clin Nutr 12:321-8 (1993); Blok et al., Eur J Clin Invest 27:1003-8 (1997); Meydani et al., J Clin Invest 92:105-13 (1993) and Molvig et al., Scand J Immunol 34:399-410 (1991)) only that by Meydani et al demonstrated an inhibitory effect of fish oil on TNF-α production. However, in this latter study fish oil was given to subjects consuming a low fat diet. In this dietary situation competition between n-6 PUFAs from the diet and n-3 PUFAs from the supplement for incorporation into cell structure would have been less than in the present study.

The results of the present study, taken with those of other studies performed on the effects of fish oil supplements on TNF-α production, indicate that the interaction of n-3 PUFA intake and cytokine biology is complex. While the dose of fish oil that is given may be a determinant of whether a suppressive effect of the oil on TNF-α production can be demonstrated at a whole population level, our data suggest that differing sensitivities of individuals to the effects of fish oil, due to genetic variation encoded by, or associated with, the TNF-α −308, LT-α +252 and IL-6 −174 SNPs, may limit the effectiveness of moderate doses of fish oil as an anti-inflammatory agent. The greater understanding of the precise nature of the determinants of sensitivity to fish oil provided by the present application will enable fish oil supplementation to be used for influencing inflammation with greater precision than is presently the case.

1. A method for assessing the susceptibility of an individual to treatment of an inflammatory disease with fish oil, comprising determining the genotype of the individual in relation to polymorphisms at the TNF-α −308, LT-α +252 and/or IL-6 −174 alleles; and inferring therefrom whether said individual responds well to treatment with fish oil.

2. A method according to claim 1, further comprising determining the inherent TNF-α status of the individual.

3. A method according to claim 1 wherein the genotype at the LT-α +252 and IL-6 −174 alleles is determined.

4. A method according to claim 1 wherein the genotype at the LT-α gene is heterozygous TNFB1/2.

5. A method according to claim 1 wherein the genotype at the IL-6 gene is homozygous GG.

6. A method according to claim 1 wherein the genotype at the LT-α and IL-6 genes is heterozygous TNFB1/2 and IL-6 GG respectively.

7. A method of assessing the susceptibility of an individual to treatment of an inflammatory disorder with fish oil, comprising:
a) determining the inherent TNF-α status of the individual; and
b) determining the genotype of the individual in relation to polymorphisms at the TNF-α −308, LT-α +252 and/or IL-6 −174 alleles; and
inferring therefrom whether said individual responds well to treatment with fish oil.

8. A method for the treatment of an inflammatory disease in a patient, which comprises assessing the susceptibility of an individual to treatment of an inflammatory disease with fish oil, said assessment comprising:
a) determining the genotype of the individual in relation to polymorphisms at the TNF-α−308, LT-α +252 and/or IL-6 −174 alleles; and
b) inferring therefrom whether said individual responds well to treatment with fish oil; and
treating said individual with an appropriate amount of fish oil.

9. A method according to claim 8 further comprising determining the inherent TNF-α status of the individual.

10. A method according to claim 1 in which the inflammatory disorder is selected from the group consisting of an inflammatory skin disorder, Perianal Crohn's disease or arthritis.


SEQUENCE LISTING (11 sequences)

SEQ ID NO: 1 - DNA, 20 bp, Artificial sequence
  Synthetic sequence Primer TNF308 common
tctcggtttcttctccatcg

SEQ ID NO: 2 - DNA, 20 bp, Artificial sequence
  Synthetic sequence Primer TNF308G
ataggttttgaggggcatgg

SEQ ID NO: 3 - DNA, 20 bp, Artificial sequence
  Synthetic sequence Primer TNF308A
ataggttttgaggggcatga

SEQ ID NO: 4 - DNA, 21 bp, Artificial sequence
  Synthetic sequence Primer LT252 common
agatcgacagagaaggggaca

SEQ ID NO: 5 - DNA, 21 bp, Artificial sequence
  Synthetic sequence Primer LT252G
cattctctgtttctgccatgg

SEQ ID NO: 6 - DNA, 21 bp, Artificial sequence
  Synthetic sequence Primer LT252A
cattctctgtttctgccatga

SEQ ID NO: 7 - DNA, 21 bp, Artificial sequence
  Synthetic sequence Primer IL6174 common
tttgttggagggtgagggtgg

SEQ ID NO: 8 - DNA, 22 bp, Artificial sequence
  Synthetic sequence Primer IL6174G
ttccccctagttgtgtcttgcg

SEQ ID NO: 9 - DNA, 22 bp, Artificial sequence
  Synthetic sequence Primer IL6174C
ttccccctagttgtgtcttgcc

SEQ ID NO: 10 - DNA, 19 bp, Artificial sequence
  Synthetic sequence Control Primer 63
tgccaagtggagcacccaa

SEQ ID NO: 11 - DNA, 20 bp, Artificial sequence
  Synthetic sequence Control Primer 64
gcatcttgctctgtgcagat